(19) United States
(12) Patent Application Publication
(10) Pub. No.: US 2004/0054286 A1
(43) Pub. Date: Mar. 18, 2004

(54) Ultrasonic transducing probe with liquid flow-through capability and related automated workstation and methods of using same

(76) Inventors: Vaughn A Audain, Durham, NC (US); Charles Amich Buckner lll, Durham, NC (US); Hendrick Mario Geysen, Charlottesville, VA (US); James R. Ormand, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/399,927

(22) PCT Filed: Nov. 5, 2001
(86) PCT No.: PCT/US01/44451

(51) Int. Cl. A61B 8/00

(52) U.S. Cl. 600/449

(57) ABSTRACT

A hollow probe cooperates with an ultrasonic transducing device designed with liquid flow-through capability. The probe and transducing device are combined into a probe assembly, which can be integrated into an automated liquid handling workstation. As a functional component of the workstation, the probe can be connected to and manipulated by a robotic arm of the workstation, and thus programmed to move in three-dimensional space to and from various locations of the sampling apparatus. In particular, the probe can be inserted into the individual wells or test tubes of a plate or rack utilized to contain sample substances. The probe can be used to conduct a variety of liquid handling tasks and additionally can be used to ultrasonically excite sample substances contained in the individual wells of the plate, thereby improving dilution of such sample substances and increasing throughput of any given sample preparation procedure. A liquid level detection device can be connected to the probe assembly.

### TECHNICAL FIELD

The present invention generally relates to an ultrasonic probe adapted for use with an automated workstation, and methods for using the probe in liquid handling and analytical processes. In particular, the present invention relates to an ultrasonic probe having liquid flow-through properties which is adapted to cooperate with a programmable robotic arm.

### BACKGROUND ART

As part of many compound generation and screening processes, small quantities of compound, in the form of powder or particles, are often deposited onto the well surfaces of plates such as microtitre and deep-well plates. Modern processes usually require the use of 96-well plates and are carried out in an automated manner at a workstation having robotic liquid-handling capabilities. The deposited solids often must be dissolved in preparation of a further task such as liquid chromatography analysis. In a time-consuming process, exact portions of solvent are added to each well of a microtitre plate. Each plate is then sealed to prevent evaporation, although it has been observed by those skilled in the art that evaporation is still a problem which can reduce the accuracy of analytical results. In order to successfully implement a high-throughput screening process, the researcher may prepare as many as fifty plates at a time. To ensure that the solids deposited in the well are completely dissolved, it is often required that the entire microtitre plate be ultrasonically excited for a period of time after the addition of solvent. Unfortunately, this process of gross or bulk sonication of the entire plate requires a large power output and tends to raise the temperature of both the plate and its contents. As a result, gross sonication bears an unacceptable risk that the compounds become damaged or chemically altered to a degree such that the compounds are rendered useless or the ensuing analytical tasks become unverifiable or inaccurate. Also, the power delivered to each individual well may differ when the bulk sonication approach is employed, resulting in non-uniform heating of the samples. Moreover, the ratio of solvent to compound must be kept high to prevent precipitation, which can lead to less accurate analytical results.

Another approach toward ensuring complete dissolution has been to manually agitate the plate. The disadvantages of this approach, including the time, imprecision and human effort required, are readily acknowledged by those skilled in the art.

Accordingly, those skilled in the art will appreciate the need for an apparatus which reduces the amount of time required to dissolve solid-phase compound samples in wells, reduces the amount of solvent evaporation, permits a higher concentration of compound within wells, enables sonic excitation to be effected in the individual wells of a plate, enables sonication to be performed at a lower power level, and enables individual analyte samples to be aspirated immediately after dissolution. Such an apparatus would advantageously result in relatively little sample temperature rise, increased automation of the screening or liquid handling process, elimination of compound damage, and would therefore yield a more accurate and repeatable analysis. The present invention, as described hereinafter in the context of exemplary embodiments and processes, is provided to meet these needs.

### DISCLOSURE OF THE INVENTION

The present invention generally provides a hollow probe which cooperates with an ultrasonic transducing device designed with liquid flow-through capability. The probe and transducing device are combined into a probe assembly which is advantageously adapted to be integrated into an automated liquid handling or sampling apparatus or workstation. As a functional component of the workstation, the probe can be connected to and manipulated by a robotic arm of the workstation, and thus programmed to move in three-dimensional space to and from various locations of the sampling apparatus. In particular, the probe can be inserted into the individual wells or test tubes of a plate or rack utilized to contain sample substances. By the design of the present invention, the probe can be used to conduct a variety of liquid handling tasks and additionally can be used to ultrasonically excite sample substances contained in the individual wells of the plate, thereby improving dilution of such sample substances and increasing throughput of any given sample preparation procedure.

According to one aspect of the present invention, an apparatus is provided for use as part of a fluid handling system and is adapted for selectively ultrasonically exciting drug, compound or chemical containing samples provided in the form of liquids, suspensions, wetted compounds, solutions or emulsions. The apparatus comprises a movable robotic assembly and an ultrasonic transducer probe assembly attached to the robotic assembly. The probe assembly includes an ultrasonic transducer body defining an internal fluid conduit and an elongate hollow probe member defining an internal bore. The probe member is disposed in mechanical communication with the transducer body to enable vibratory energy to be transferred from the transducer body to the probe member. The internal bore of the probe member fluidly communicates with the internal conduit of the transducer body, and terminates at an orifice defined by the probe member.

According to another aspect of the present invention, the probe assembly includes a housing attached to the robotic assembly, and the transducer body is disposed in the housing.

According to an additional aspect of the present invention, the probe member has a sharpened tip adapted to puncture a closure provided with a substance container.

According to a further aspect of the present invention, an apparatus has liquid flow-through capability and is adapted to sonicate a drug, compound or chemical containing substance, and is further adapted to detect the level of the substance in a container. The apparatus comprises an ultrasonic transducer probe assembly and a liquid level detection device electrically connected to the probe assembly. The ultrasonic transducer probe assembly includes an ultrasonic transducer body defining an internal fluid conduit and an elongate hollow probe member defining an internal bore. The probe member is disposed in mechanical communication with the transducer body to enable vibratory energy to be transferred from the transducer body to the probe member. The internal bore fluidly communicates with the internal conduit and terminates at an orifice defined by the probe member.

According to yet another aspect of the present invention, a fluid handling workstation is provided, and is adapted to perform sonication tasks in individual wells of well-containing plates. The workstation comprises a workstation frame including a lateral track; a robotic assembly movable along the lateral track; and an ultrasonic transducer probe assembly attached to the robotic assembly. The probe assembly includes an ultrasonic transducer body defining an internal fluid conduit and an elongate hollow probe member defining an internal bore. The probe member is disposed in mechanical communication with the transducer body to enable vibratory energy to be transferred from the transducer body to the probe member. The internal bore of the probe member fluidly communicates with the internal conduit of the transducer body, and terminates at an orifice defined by the probe member.

According to still another aspect of the present invention, the robotic assembly includes a vertical arm defining a vertical track and a horizontal arm defining a horizontal track. The probe assembly engages the vertical arm and is movable along the vertical track, the vertical arm engages the horizontal arm and is movable along the horizontal track, and the horizontal arm is movable along the lateral track of the workstation frame.

According to a further aspect of the present invention, the workstation comprises an injection port accessible by the probe member. The injection port includes an annular sealing member adapted to receive the probe member therethrough and an injection bore adapted to receive the probe member therein. The sealing member is disposed in an internal sealing region defined by the injection port.

According to a still further aspect of the present invention, the workstation comprises a rinse station accessible by the probe member. The rinse station includes a main body and an adapter fitting attached to the main body. The main body defines a rinsing bore adapted to receive the probe member therein. The adapter fitting has an aperture fluidly communicating with the rinsing bore and is adapted to receive the probe member therethrough. The aperture is sized to ensure that the probe member does not contact the main body when inserted into the rinsing bore.

The probe assembly and/or workstation provided in accordance with the present invention can operate in conjunction with a number of other devices or instruments employed in liquid handling and sample preparation tasks. Such devices and instruments include, without limitation, a dilution device, a syringe pump, chromatography apparatus, and the like.

The present invention also provides a process for preparing drug, compound or chemical containing fluid samples for subsequent analysis. An automated support assembly and an ultrasonic transducer probe assembly are provided. The ultrasonic transducer probe assembly is attached to the support assembly, and includes an ultrasonic transducer body defining an internal fluid conduit and an elongate hollow probe member defining an internal bore. The probe member is disposed in mechanical communication with the transducer body to enable vibratory energy to be transferred from the transducer body to the probe member. The internal bore fluidly communicates with the internal conduit and terminates at an orifice defined at a tip of the probe member. A plate including a plurality of wells is provided. One or more of the wells contain a drug, compound or chemical substance. The support assembly transports the probe assembly to the plate and lowers the probe member into a selected one of the wells of the plate. The sample substance is at least partially diluted by causing a volume of solvent to flow through the internal fluid conduit of the ultrasonic transducer body of the probe assembly, through the internal bore of the probe member, out from the orifice of the probe member, and into the selected well of the plate. The sample substance is diluted by activating the probe assembly to transfer vibratory energy to the sample substance from the tip of the probe member. The addition of solvent and sonication of sample substance can be repeated for each well of the plate, and well as for any other plates provided. Moreover, a quantity of sonicated sample substance can be withdrawn from the well into the probe member, so that this quantity can be transported to another location such as an injection port, a rinse station, or another plate.

The present invention also provides a sonicated sample substance prepared in accordance with the above-disclosed process.

The present invention further provides a sonicated organic tissue sample prepared in accordance with the above-disclosed process.

It is therefore an object of the present invention to provide an ultrasonic transducer device in which a probe can transmit vibratory energy to a substance to improve dissolution thereof, as well as aspirate and dispense the substance.

It is also an object of the present invention to provide an ultrasonic transducer device adapted to transport solvent to a sample substance, especially a substance contained in a well of a plate, sonicate the substance, and then immediately aspirate the substance from the well if desired.

It is another object of the present invention to provide an automated liquid handling workstation which includes the novel ultrasonic transducer device instead of a more conventional sampling needle.

It is yet another object of the present invention to provide a rinse station for a liquid handling apparatus which is adapted to receive the novel ultrasonic transducer device.

It is still another object of the present invention to provide an injection port, especially of the type employed in conjunction with chromatography equipment, which is adapted to receive the novel ultrasonic transducer device.

It is a further object of the present invention to provide a process for preparing sample substances using the novel ultrasonic transducer device.

Some of the objects of the invention having been stated hereinabove, other objects will become evident as the description proceeds, when taken in connection with the accompanying drawings as best described hereinbelow.

### BRIEF DESCRIPTION OF THE DRAWINGS

FIG. 1 is a perspective view of an automated liquid handling apparatus in which an ultrasonic transducing probe and related components have been integrated in accordance with the present invention;

FIG. 2 is another perspective view of the liquid handling apparatus illustrated in FIG. 1 wherein the plates have been removed for clarity of description;

FIG. 3 is a perspective view of an ultrasonic transducing probe assembly mounted to a robotic arm of the liquid handling apparatus illustrated in FIG. 1;

FIG. 4 is a perspective, partially cutaway view of an adapter housing in which an ultrasonic transducing probe is mounted in accordance with the present invention;

FIG. 5 is an exploded view of the ultrasonic transducing probe assembly illustrated in FIG. 3;

FIG. 6 is a cutaway view of the ultrasonic transducing probe provided in accordance with the present invention;

FIG. 6A is a cutaway view of an alternative ultrasonic transducing probe provided in accordance with the present invention;

FIG. 7 is a partially cutaway view of the ultrasonic transducing probe inserted into an injection port in accordance with the present invention;

FIG. 8 is an exploded view of the ultrasonic transducing probe and injection port illustrated in FIG. 7;

FIG. 9 is an exploded, cutaway view of the injection port illustrated in FIG. 7;

FIG. 10 is a cutaway view of the ultrasonic transducing probe inserted into a rinse station port in accordance with the present invention; and

FIG. 11 is a schematic view of the ultrasonic transducing probe operating in conjunction with a liquid level detection device in accordance with the present invention.

### DETAILED DESCRIPTION OF THE INVENTION

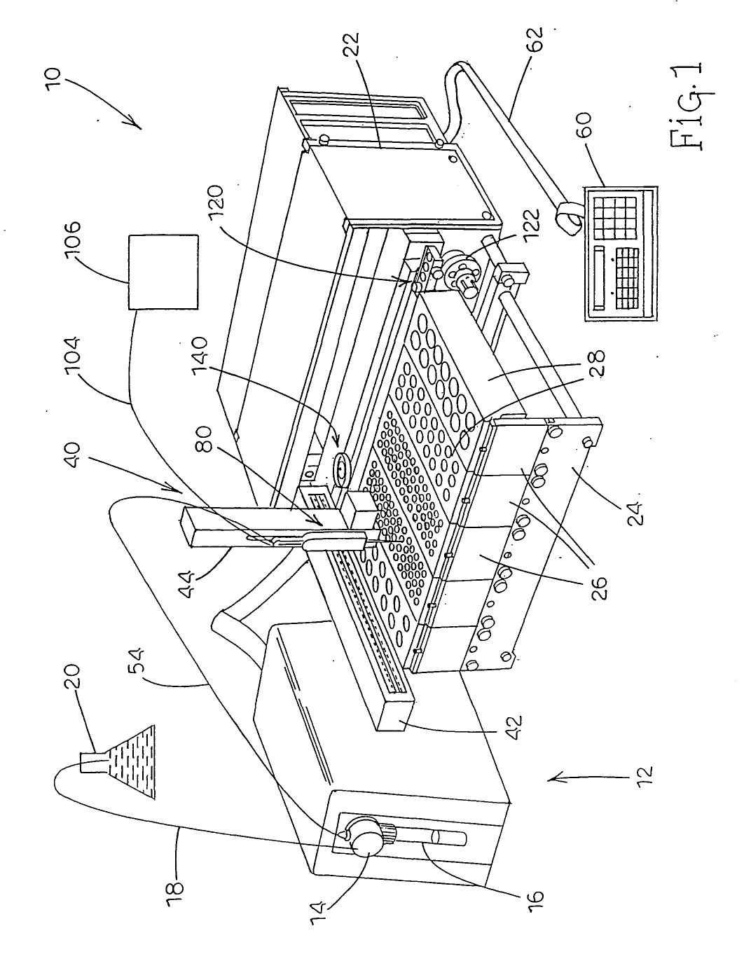

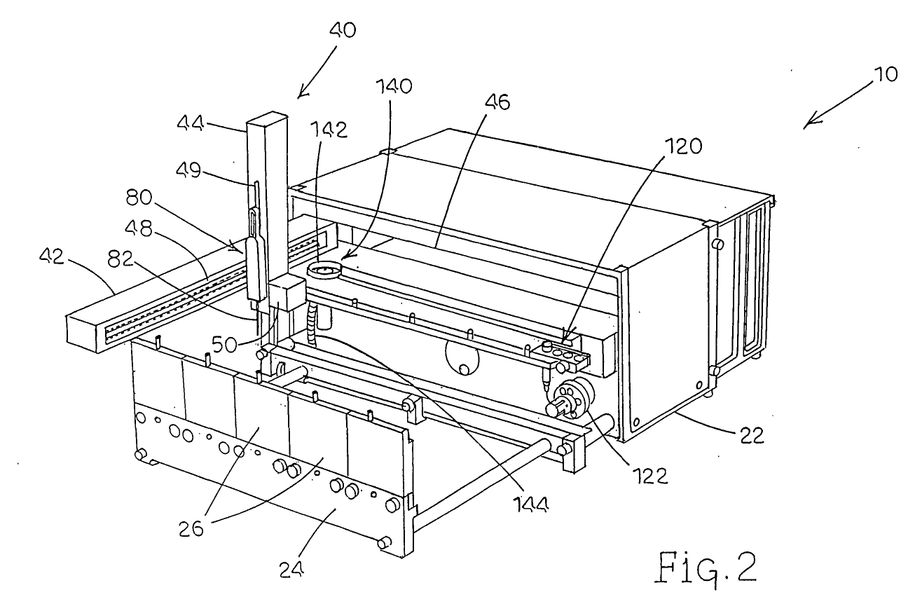

Referring now to FIGS. 1 and 2, an automated liquid handling or sampling apparatus, generally designated 10, is illustrated in accordance with the present invention. In the exemplary, inventive embodiment illustrated herein, sampling apparatus 10 can be a modified version of a commercially available GILSON™ apparatus, of which various models are available from Gilson Medical Electronics, Inc. For example, a GILSON™ Model No. 215 platform has been found to be suitable in the practice of the present invention. Other apparatuses or platforms which could be adapted to operate in conjunction with the present invention include ZYMARK™ and PACKARD™ models. It will be understood, however, that automated sampling apparatus 10 provided in accordance with the present invention can be constructed from fully original components. Thus, the apparatus depicted in FIGS. 1 and 2 is intended herein to represent either a fully original embodiment or a commercially available platform modified or adapted in accordance with the present invention. Sampling apparatus 10 is generally used for sample preparation, and is capable of being programmed by means of written software to perform a wide variety of liquid handling and preparation tasks. For example, sampling apparatus 10 can be equipped with an electrical input/output interface (not shown) to enable communication with a suitable liquid or gas chromatography analysis device if desired.

Sampling apparatus 10 ordinarily includes a dilution module, generally designated 12, which controls the movement of liquid within various points of sampling apparatus 10. A valve 14 is mounted to dilution module 12, and a syringe 16 depends therefrom. As is well known in the art, a movable boundary is disposed within syringe 16 and is actuated by a stepper motor and associated drive unit (not shown) to provide both aspiration and positive pressure to the various fluid conduits associated with sampling apparatus 10. The actuation may be programmed into sampling apparatus 10. A length of solvent inlet tubing 18, preferably formed of PTFE, is connected to valve 14 to supply rinse solvent to sampling apparatus 10 from a solvent reservoir 20. Examples of solvents commonly used include methanol, ethanol, water, acetonitrile, acetone, isopropanol, hexane, diethyl ether, and toluene.

Sampling apparatus 10 generally includes a main frame 22. A plate holder 24 is attached to main frame 22 and includes a series of adapter plates 26. As shown in FIG. 1, a plurality of plates 28 may be mounted on plate holder 24 by means of alignment with adapter plates 26. A wide variety of plates 28, such as microtitre plates, deep-well plates and test tube racks, are available depending on the desired application. Each plate 28 includes an array of wells for containing reagents, compounds, samples of liquid substances, and the like, or includes holes for holding vials, test tubes or other vessels of differing sizes for this purpose.

As best shown in FIG. 2, also attached to main frame 22 is a movable robotic assembly, generally designated 40. Robotic assembly 40 includes a horizontal arm 42 and a vertical arm 44. Horizontal arm 42 is slidably carried on a track 46 mounted within main frame 22. An additional track 48 is formed on horizontal arm 42, in which vertical arm 44 is slidably carried. One or more stepper motors and associated drives (not shown) disposed within main frame 22, or on vertical arm 44 as in the case of motor 50, provide actuation for robotic assembly 40 along a three-axis coordinate system. As in the case of dilution module 12, this actuation may be controlled by software interfacing with sampling apparatus 10. In the conventional form of sampling apparatus 10, a sampling needle (not shown) would be movably mounted to a vertically disposed track 49 of vertical arm 44 and employed to load and extract liquid substances to and from different positions over and proximate to plates 28 shown in FIG. 1. A length of transfer tubing 54 (see FIG. 1), preferably formed of PTFE, would provide fluid communication between this sampling needle and valve 14 of dilution module 12. In the present invention, however, as shown in FIGS. 1 and 2, an ultrasonic transducing probe assembly, generally designated 80, equipped with a probe 82 having liquid flow-through capability has been substituted in the place of a conventional sampling needle.

Through the movement of horizontal arm 42, vertical arm 44 and ultrasonic probe assembly 80, probe 82 according to the present invention may be programmed to accomplish not only sonication tasks, but also chromatography injection, and a variety of liquid handling and sample preparation tasks such as transferring solvent to vials and/or wells disposed in plates 28 and transferring liquid substances from one vial or well to another vial or well. A remote keypad or computer 60 (see FIG. 1) may be connected to sampling apparatus 10 via a ribbon cable 62 and used for entering instructions into memory, recalling previously written programs, and otherwise controlling the operation of sampling apparatus 10, including robotic assembly 40 and ultrasonic probe assembly 80.

Sampling apparatus 10 also includes an injection port, generally designated 120, which is accessible by probe 82. Injection port 120 fluidly communicates with an injection valve 122, and is used to deliver samples to a high-pressure liquid chromatography (HPLC) device or gas chromatography device (not shown) if desired. Sampling apparatus 10 further includes a rinsing station, generally designated 140, which may be used for eliminating waste products and purging the fluid paths of sampling apparatus 10 between the operative steps of an intended procedure. Rinsing station 140 includes a trough or a cup 142, which is also accessible by probe 82, and a drain tube 144 (see FIG. 2).

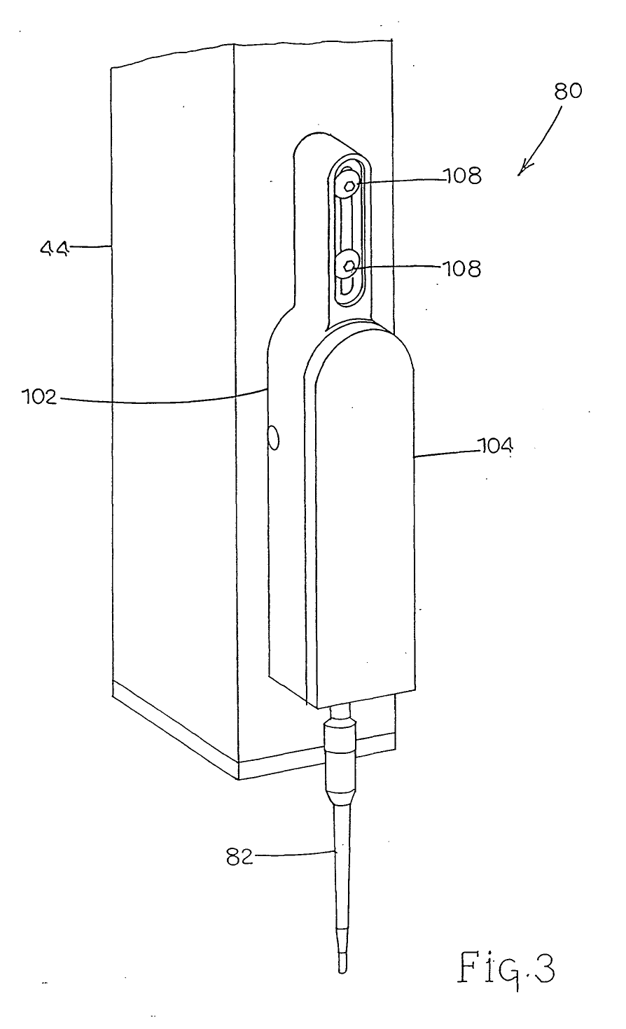

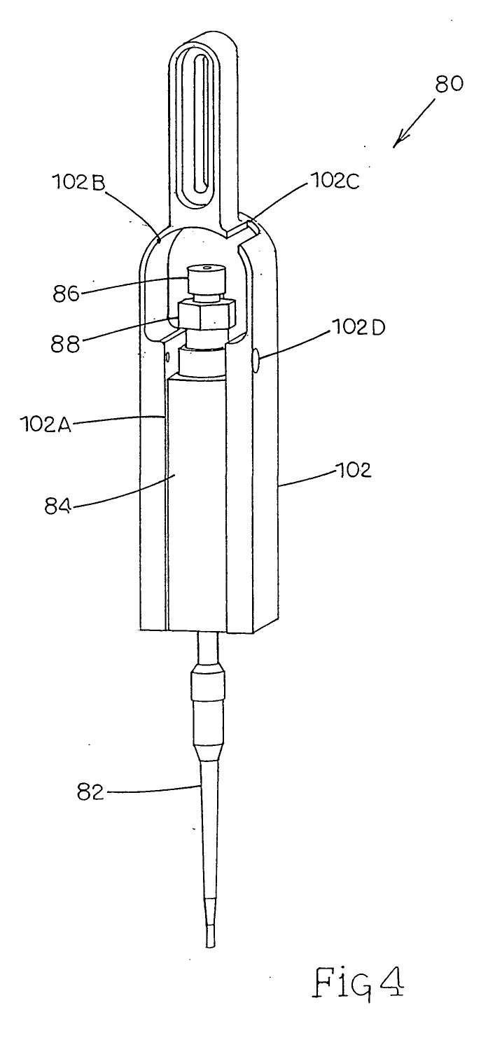

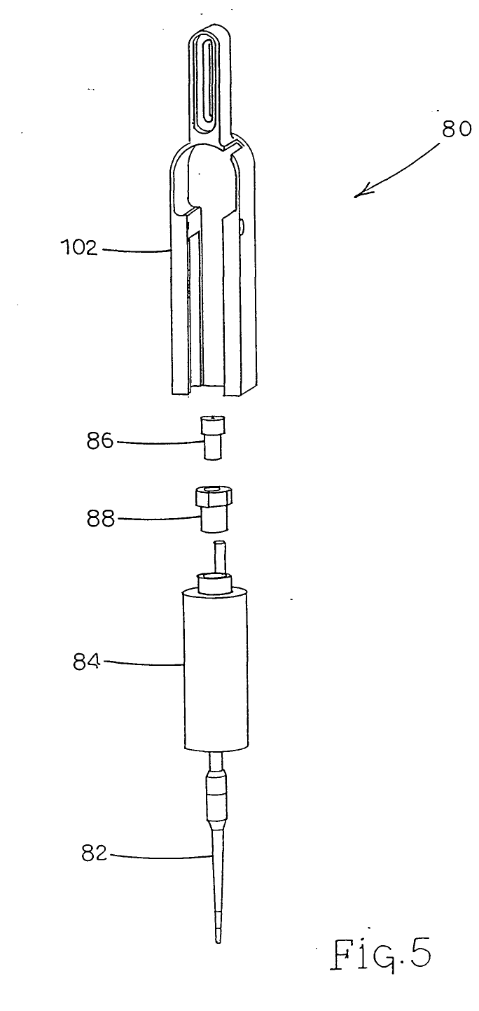

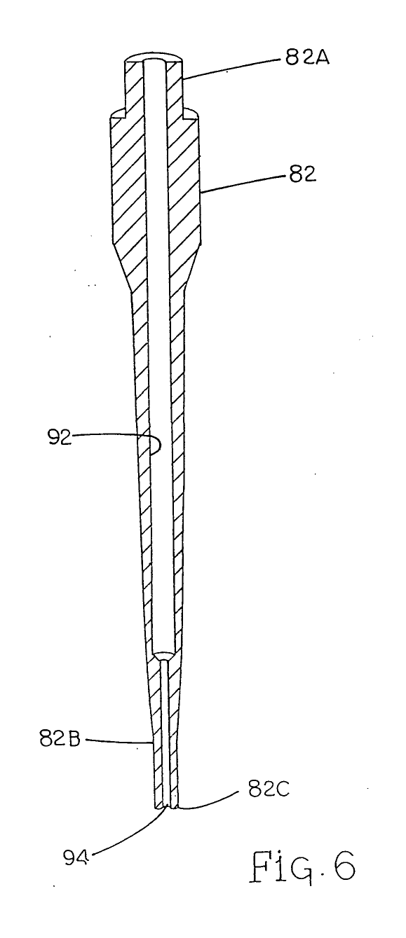

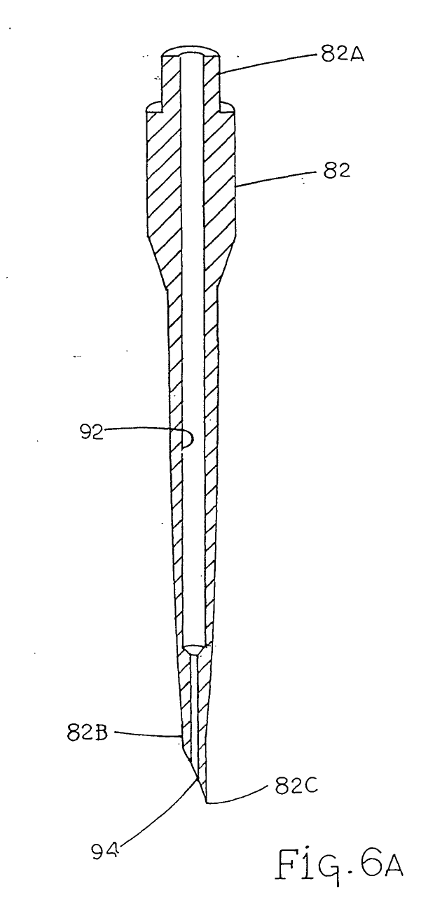

Referring now to FIGS. 3-6, ultrasonic transducing probe assembly 80 is illustrated in more detail. Probe assembly 80 includes a ultrasonic converter body or handpiece 84, such as a MISONIX™ handpiece commercially available from Misonix Inc. of Farmingdale, N.Y., as Part No. 2325. Converter body 84 has a flow-through design, and accordingly includes an internal passage (not shown) to enable fluid to flow from transfer tubing 54, through a tubing adapter 86 and a top fitting 88, through converter body 84, and finally to probe 82. A suitable probe 82 is also available from Misonix Inc. as Part No. 1825. As best shown in FIG. 6, the body of probe includes a neck section 82A to which top fitting 88 is secured such as by threading. The outside diameter of probe 82 is reduced over one or more tapered sections. The outside diameter of a distal section 82B is small enough to permit probe 82 to be inserted into the wells of a standard-sized plate 28. Probe 82 has a hollow interior bore 92 terminating at a distal orifice 94 defined at a tip 82C of probe 82. Hollow interior bore 92 includes a reduced-diameter section within distal section 82B of probe 82. In the exemplary embodiment, orifice 94 has a 0.6 mm diameter. Probe 82 serves as an elongate horn member which transfers sonic energy to probe tip 82C. In use, when a fluid such as a solvent is pumped through converter body 84 and probe 82 with probe assembly 80 activated, a fine mist can be produced at orifice 82C. In the present embodiment, probe assembly 80 has been designed so as not to leak under operating back pressures of up to approximately 120 psi, which makes probe assembly 80 suitable for use in conjunction with liquid chromatographic injection.

An alternative version of probe 82 is illustrated in FIG. 6A. Probe tip 82C in FIG. 6A has been cut, either arcuately or at an angle such as 30E, so as to present a sharpened edge. The sharpened edge is useful in the case where a substance container such as a vial or test tube includes a closure such as a septum. The sharpened edge facilitates the penetration or puncturing of the septum by probe 82.

Referring back to FIGS. 3-5, converter body 84 fits into a probe assembly adapter or housing 102 and is protected by a removable front cover 104. Probe assembly adapter or housing 102 is preferably constructed from machined aluminum, and is shaped to accommodate converter body 84 as well as tubing adapter 86 and fitting 88. For example, an elongate chamber 102A can be formed to accommodate converter body 84, and an upper chamber 102B formed to accommodate tubing adapter 86 and fitting 88. In addition, a slot 102C is formed on probe assembly adapter 102 to accommodate fluid transfer tubing 54 (see FIG. 1) and an electrical control cable 104 to pass therethrough. Control cable 104 is attached between converter body 84 and a remote generator device or base station 106 so that base station 106 (see FIG. 1) can provide electrical power to converter body 84 and thus drive the vibratory action. Another cable (not shown) can be run between base station 106 and computer 60 or other electronic device (see FIG. 1) to turn probe assembly 80 ON and OFF. Converter body 84 can be secured within probe assembly adapter 102 such as by threading a screw (not shown) into an aperture 102D of probe assembly adapter 102. As shown in FIG. 3, probe assembly adapter 102 is adapted to fit onto vertical arm 44 of robotic assembly 40 in the place of a standard sampling needle, using the same mounting boss and screws 108. Preferably, the respective lengths of probe assembly adapter 102 and probe 82 are such that probe tip 82C matches the position originally assumed by the sampling needle, and hence eliminates the need for major z-axis compensation.

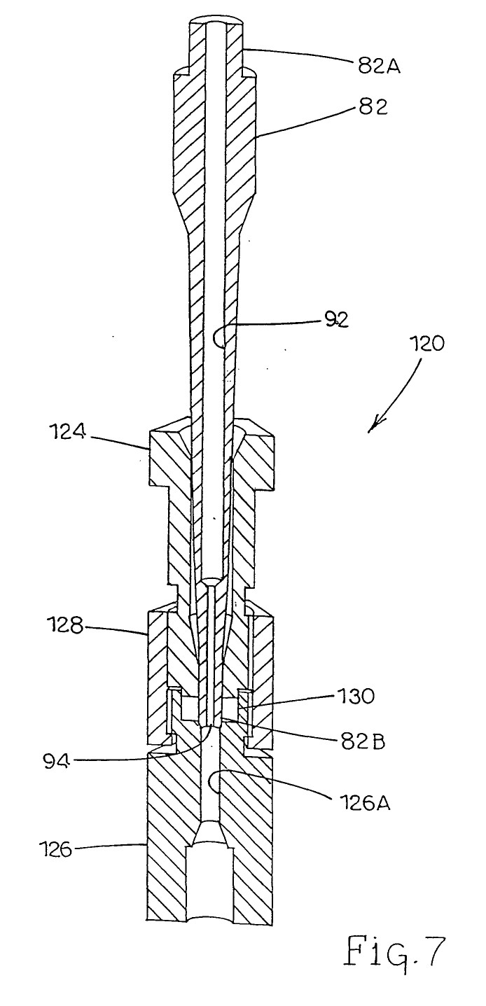

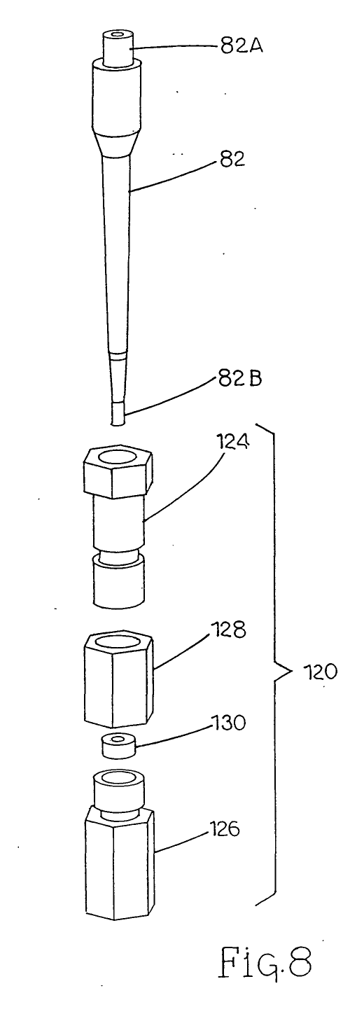

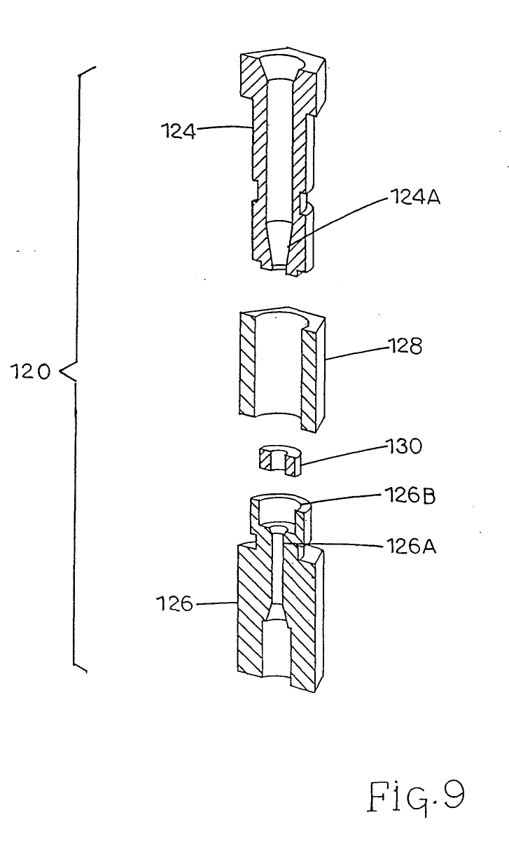

Referring to FIGS. 7-9, injection port 120 is illustrated in more detail. Injection port 120 is designed to receive probe 82 and enable probe 82 to inject sample media without leakage. Injection port 120 includes an upper body 124, a lower body 126, a collar 128, and a TEFLON7 seal 130. Upper and lower bodies 124 and 126 may be secured together by providing mating threads on upper and lower bodies 124 and 126 and on collar 128. Upper body 124 has a tapering inside surface 124A (see FIG. 9) to accommodate probe 82. Lower body 126 includes an internal flow-through bore 126A with which tip 82C of probe 82 makes contact. As shown in FIG. 7, tip 82C and internal bore 126A preferably have complementary tapered or chamfered surfaces to improve their contact. An internal volume 126B of lower body 126 defines a sealing region into which seal 130 is installed. Seal 130 is generally interposed between upper and lower bodies 124 and 126 in coaxial relation to distal section 82B of probe 82, thereby filling the space of the sealing region and establishing a good seal between probe 82 and injection port 120.

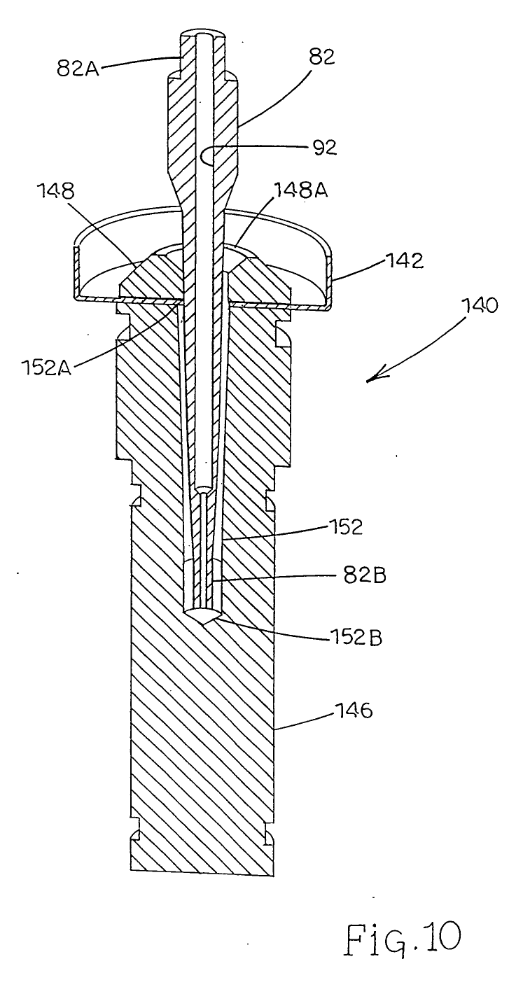

Referring now to FIG. 10, rinsing station 140 is illustrated in more detail. Rinsing station 140 includes a main body 146 and an annular adapter fitting 148 attached to main body 146 generally above cup 142. Main body 146 includes a rinsing bore 152 having an open end 152A communicating with an aperture 148A of fitting 148 and a closed end 152B terminating at a point within main body 146. Rinsing bore 152 may be tapered to accommodate probe 82. The diameter of aperture 148A is sized relative to rinsing bore 152 such that when probe 82 is inserted through aperture 148A into rinsing bore 152, the outer surfaces of probe 82 are close to but not touching rinsing bore 152. Rinsing station 140 is thus designed to receive probe 82 therein so that cleaning solvent can be aspirated through probe 82 and its orifice 94, and conducted through rinsing bore 152 so that both the inner and outer surfaces of probe 82 are contacted by the cleaning solvent and cleaned thereby.

An exemplary operation of probe 82 as integrated into sampling apparatus 10 will now be described, with general reference being made to all Figures disclosed herein. Plates 28 such as microtitre plates containing samples of dry compound in one or more wells (or, alternately, racks supporting a plurality of test tubes, vials or other substance containers) are placed into plate holder 24. Depending on the particular application, the respective labels or identifications of the compounds, their coordinate positions in 96-well plate 28, their respective masses, and the positions of plates 28 in plate holder 24 can be inputted into computer 60 as part of the programming of tasks to be performed by robotic assembly 40 and dilution module 12. Also, it may be desired to initially cause robotic assembly 40 to transport probe assembly 80 to rinse station 140 and insert probe 82 therein, and to cause dilution module 12 to draw a volume of rinse solvent from reservoir 20 and pump the solvent through probe 82 in order to flush the fluid lines and pre-clean probe 82. Upon activation of sampling apparatus 10, robotic assembly 40 transports probe assembly 80 to the wells of one or more plates 28, lowers probe 82 into individual wells, and dispenses a controlled amount of solvent or other fluid through probe 82 into each well. If desired, sampling apparatus 10 may be programmed to mix two or more different types of solvents in a given well in order to create binary, tertiary, quaternary, etc. solvent systems. If a closure such as a septum exists, probe 82 is capable of puncturing such a barrier.

Prior to sonication, residual solvent can be removed from probe 82 by drawing air in order to prevent excessive or unwanted dilution of a sample. At each well, probe 82 is then caused to make contact with the wetted substance contained therein, and probe assembly 80 is activated to transfer vibrational energy to tip 94 of probe 82 and thereby sonicate the substance for a predetermined period of time (e.g., a few seconds). The primary function of the sonication process in the exemplary embodiment is to effect complete dissolution of the dry compound contained in a given well. However, in the appropriate situation, vibrating probe 82 could be operated long enough to deliberately cause a rise in sample temperature.

After sonication, probe 82 is further employed to aspirate a predetermined quantity of sample of the dissolved compound. Robotic assembly 40 can then transport probe assembly 80 to a variety of locations of sampling apparatus 10, depending on the particular procedure being undertaken. For instance, probe assembly 80 can move to another plate 28 containing wells or holding test tubes, and the aspirated sample can be dispensed through probe tip 94 into the wells or test tubes as part of some analytical or combinatorial process. In addition, probe assembly 80 can move to injection port 120 and probe 82 inserted therein as shown in FIG. 7, and the aspirated sample can then be provided for analysis in a liquid chromatograph, for example. It is possible to provide more than one chromatograph, such that multiple analyses can occur simultaneously to thereby increase throughput. It is also possible to provide a port or other probe receiving means which fluidly connects probe to a gas chromatograph. Finally, probe assembly 80 can be moved to rinse station 140 and probe 82 installed therein as shown in FIG. 10, so that probe 82 can be cleaned to prevent cross-contamination.

Sampling apparatus 10 can be programmed to execute one or more of the above-described process steps for each well of one or more plates 28 in a repeatable, cyclical process.

As an additional application of the present invention, plates 28 could be constructed from a translucent or transparent material such as quartz, to enable the examination of optical and spectral properties of the substances residing in each well. Sample substances could be initially provided in a quartz microtitre plate, or probe assembly 80 could be employed to transport samples to the quartz plate from another type of plate.

In another, more specific application of the present invention, tissue samples can be homogenized in preparation for RNA extraction. In this application, tissue samples are added to the wells of plate 28 or to individual containers such as EPPENDORFJ tubes held in a suitable plate 28. In the latter case, plate 28 is provided in the form of a rack adapted to hold such tubes. After the addition of a suitable solvent to a given tissue sample, the sonication carried out by probe 82 generally occurs over a period longer than a few seconds, for example approximately 30 to 60 seconds, in order to adequately break up and dissolve the tissue sample. Probe 82 is then used to aspirate and deliver a predetermined quantity of the dissolved tissue sample to another container for further processing. This other container may be mounted at sampling apparatus 10.

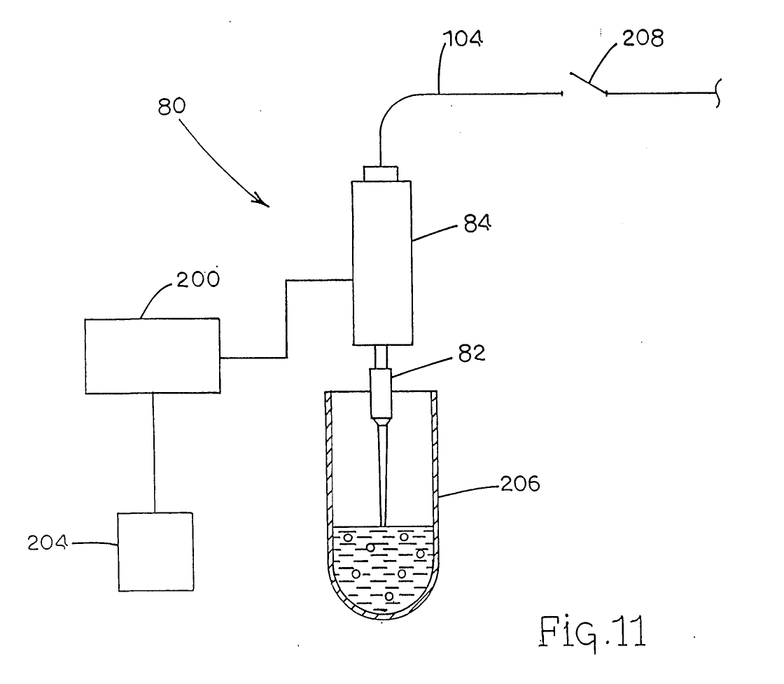

Referring now to FIG. 11, a liquid level detection task can be made a part of the various processes involving the use of probe 82. A liquid level detection device 200, such as a conventional capacitive-type transducer, is electrically coupled to probe assembly 80 such as at converter body 84. Liquid level detection device 200 is also connected to some type of readout or display 204, which may be part of an electronic device separate from or integrated into sampling apparatus 10 of FIGS. 1 and 2. When probe 82 is inserted into a well or other container, represented in FIG. 11 as 206, and contacts the solution or suspension contained therein, device 200 can measure the level of liquid in container 206. An ON/OFF switch 208 can be provided on control cable 104 to isolate the operation of liquid level detection device 200 and thereby ensure its accuracy. Liquid level detection device 200 correlates the level of solution or suspension in container 206 to a sensed measurement of capacitance, and readout 204 displays an indication of that level.

It will be understood that various details of the invention may be changed without departing from the scope of the invention. Furthermore, the foregoing description is for the purpose of illustration only, and not for the purpose of limitation—the invention being defined by the claims.

What is claimed is:

1. An apparatus adapted for use as part of a fluid handling system and adapted for selectively ultrasonically exciting drug, compound or chemical containing samples provided in the form of liquids, suspensions, wetted compounds, solutions or emulsions, the apparatus comprising:
(a) a movable robotic assembly; and
(b) an ultrasonic transducer probe assembly attached to the robotic assembly, the probe assembly including an ultrasonic transducer body defining an internal fluid conduit and an elongate hollow probe member defining an internal bore, the probe member disposed in mechanical communication with the transducer body to enable vibratory energy to be transferred from the transducer body to the probe member, the internal bore fluidly communicating with the internal conduit and terminating at an orifice defined by the probe member.

2. The apparatus according to claim 1 wherein the robotic assembly includes a vertical arm defining a vertical track and a horizontal arm defining a horizontal track, and the probe assembly engages the vertical arm and is movable along the vertical track, the vertical arm engages the horizontal arm and is movable along the horizontal track, and the horizontal arm is movable along a lateral direction.

3. The apparatus according to claim 1 wherein the probe assembly includes a housing attached to the robotic assembly, and the transducer body is disposed in the housing.

4. The apparatus according to claim 1 wherein the transducer body is disposed in electrical communication with a power source.

5. The apparatus according to claim 1 wherein the internal fluid conduit of the transducer body fluidly communicates with a reservoir over a fluid transfer line.

6. The apparatus according to claim 1 wherein the internal fluid conduit of the transducer body fluidly communicates with a fluid aspirating and pumping unit over a fluid transfer line.

7. The apparatus according to claim 6 wherein the fluid aspirating and pumping unit is part of a dilution device.

8. The apparatus according to claim 6 wherein the fluid aspirating and pumping unit includes a syringe-type pump.

9. The apparatus according to claim 6 wherein the fluid aspirating and pumping unit communicates with a reservoir over a fluid supply line.

10. The apparatus according to claim 1 wherein the probe member terminates at an acute probe tip adapted to penetrate a closure.

11. An apparatus having liquid flow-through capability and adapted to sonicate a drug, compound or chemical containing substance and to detect the level of the substance in a container, the apparatus comprising:
(a) an ultrasonic transducer probe assembly including an ultrasonic transducer body defining an internal fluid conduit and an elongate hollow probe member defining an internal bore, the probe member disposed in mechanical communication with the transducer body to enable vibratory energy to be transferred from the transducer body to the probe member, the internal bore fluidly communicating with the internal conduit and terminating at an orifice defined by the probe member; and
(b) a liquid level detection device electrically connected to the probe assembly.

12. The apparatus according to claim 11 comprising a movable robotic assembly, wherein the ultrasonic transducer probe assembly is attached to the robotic assembly.

13. A fluid handling workstation adapted to perform sonication tasks in individual wells of well-containing plates, the workstation comprising:
(a) a workstation frame including a lateral track;
(b) a robotic assembly movable along the lateral track; and
(c) an ultrasonic transducer probe assembly attached to the robotic assembly, the probe assembly including an ultrasonic transducer body defining an internal fluid conduit and an elongate hollow probe member defining an internal bore, the probe member disposed in mechanical communication with the transducer body to enable vibratory energy to be transferred from the transducer body to the probe member, the internal bore fluidly communicating with the internal conduit and terminating at an orifice defined by the probe member.

14. The workstation according to claim 13 wherein the robotic assembly includes a vertical arm defining a vertical track and a horizontal arm defining a horizontal track, and the probe assembly engages the vertical arm and is movable along the vertical track, the vertical arm engages the horizontal arm and is movable along the horizontal track, and the horizontal arm is movable along the lateral track of the workstation frame.

15. The workstation according to claim 13 wherein the probe assembly includes a housing attached to the robotic assembly, and the transducer body is disposed in the housing.

16. The workstation according to claim 13 comprising a plate removably mounted at the workstation and including an array of wells accessible by the probe member.

17. The workstation according to claim 13 comprising an injection port accessible by the probe member, the injection port including an annular sealing member adapted to receive the probe member therethrough and an injection bore adapted to receive the probe member therein, the sealing member disposed in an internal sealing region defined by the injection port.

18. The workstation according to claim 13 comprising a rinse station accessible by the probe member, the rinse station including a main body and an adapter fitting attached to the main body, the main body defining a rinsing bore adapted to receive the probe member therein, the adapter fitting having an aperture fluidly communicating with the rinsing bore and adapted to receive the probe member therethrough, the aperture sized to ensure that the probe member does not contact the main body when inserted into the rinsing bore.

19. The workstation according to claim 13 comprising a liquid level detection device electrically connected to the probe assembly

20. The workstation according to claim 13 wherein the probe member terminates at an acute probe tip adapted to penetrate a closure.

21. A process for preparing drug, compound or chemical containing fluid samples for subsequent analysis comprising the steps of:
(a) providing an automated support assembly;
(b) providing an ultrasonic transducer probe assembly attached to the support assembly, wherein the probe assembly includes an ultrasonic transducer body defining an internal fluid conduit and an elongate hollow probe member defining an internal bore, the probe member is disposed in mechanical communication with the transducer body to enable vibratory energy to be transferred from the transducer body to the probe member, the internal bore fluidly communicating with the internal conduit and terminating at an orifice defined at a tip of the probe member;
(c) providing a plate including a plurality of containers, wherein at least some of the containers contain a drug, compound or chemical substance;
(d) causing the support assembly to transport the probe assembly to the plate and to lower the probe member into a selected one of the containers of the plate;
(e) at least partially diluting the sample substance by causing a volume of solvent to flow through the internal fluid conduit of the ultrasonic transducer body of the probe assembly, through the internal bore of the probe member, out from the orifice of the probe member, and into the selected container of the plate;
(f) sonicating the at least partially diluted sample substance by activating the probe assembly to transfer vibratory energy to the sample substance from the tip of the probe member; and
(g) repeating steps (d) through (f) at other containers of the plate.

22. The process according to claim 21 comprising the step of withdrawing a quantity of the sonicated sample substance from the selected container into the internal bore of the probe member.

23. The process according to claim 22 comprising the steps of causing the support assembly to transport the probe assembly to an additional plate and dispensing the withdrawn quantity of sonicated sample substance through the orifice of the probe member to a location of the additional plate.

24. The process according to claim 22 comprising the steps of providing an injection port including an injection bore and an annular sealing member disposed in an internal sealing region defined by the injection port, causing the support assembly to transport the probe assembly to the injection port, lowering the probe member into the injection bore and into the sealing member, and dispensing the withdrawn quantity of sonicated sample substance into the injection bore.

25. The process according to claim 21 comprising the steps of providing a rinse station including a main body defining a rinsing bore and an adapter fitting attached to the main body, the adapter fitting defining an aperture fluidly communicating with the rinsing bore, causing the support assembly to transport the probe assembly to the rinse station, lowering the probe member through the aperture of the adapter fitting and into the injection bore such that the probe member is spaced from the main body, and causing a rinse solvent to flow through the probe member into the rinsing bore, whereby inner and outer surfaces of the probe member are cleaned by the rinse solvent.

26. The process according to claim 21 comprising the step of providing a liquid handling apparatus to which the automated support assembly is movably mounted.

27. The process according to claim 21 comprising the step of using the probe member to puncture a closure of the selected container.

28. The process according to claim 21 comprising the step of using a liquid level detection device connected to the probe assembly to measure a level of the sample substance contained in the selected container.

29. A sonicated sample substance prepared according to the process of claim 21.

30. The process according to claim 21 wherein the sample substance is provided in the form of an organic tissue sample.

31. A sonicated organic tissue sample prepared according to the process of claim 30.
